(19) United States
(12) Patent Application Publication
(10) Pub. No.: US 2025/0308382 A1
(43) Pub. Date: Oct. 2, 2025

(54) VISUAL FIELD MEASUREMENT METHOD AND MOVING BODY

(71) Applicant: HONDA MOTOR CO., LTD., Tokyo (JP)

(72) Inventors: Misako Koike, Wako-shi (JP); Kosuke Nakanishi, Wako-shi (JP); Gakuyo Fujimoto, Wako-shi (JP)

(73) Assignee: HONDA MOTOR CO., LTD., Tokyo (JP)

(21) Appl. No.: 19/039,918

(22) Filed: Jan. 29, 2025

(30) Foreign Application Priority Data
Mar. 28, 2024 (JP) 2024-054470

(51) Int. Cl. G08G 1/01; B60W 50/14; G08G 1/16

(52) U.S. Cl. CPC G08G 1/0141; B60W 50/14; G08G 1/163

(57) ABSTRACT

A method for measuring a visual field of a subject includes arranging a target object at a position at a measurement target distance from the subject and arranging a symbol at a plurality of positions around the target object and determining the visual field of the subject based on a position of the plurality of positions at which the subject can visually recognize the symbol while looking at the target object.

### CROSS-REFERENCE TO RELATED APPLICATION(S)

This application claims priority to and the benefit of Japanese Patent Application No. 2024-054470, filed Mar. 28, 2024, the entire disclosure of which is incorporated herein by reference.

### BACKGROUND OF THE INVENTION

### Field of the Invention

The present invention relates to a visual field measurement method and a moving body.

### Description of the Related Art

There is a known technique that uses the visual field of a driver to provide driving assistance. Japanese Patent Laid-Open No. 2023-119428 describes that a visual field inspection is performed by irradiating a windshield with light.

### SUMMARY OF THE INVENTION

The inventors have found that the visual field of a person can vary depending on the focal length of the eye. Some aspects of the present invention measure a visual field according to a situation. According to some embodiments, a method for measuring a visual field of a subject, the method comprising: arranging a target object at a position at a measurement target distance from the subject and arranging a symbol at a plurality of positions around the target object; and determining the visual field of the subject based on a position of the plurality of positions at which the subject can visually recognize the symbol while looking at the target object is provided.

### BRIEF DESCRIPTION OF THE DRAWINGS

FIG. 1 is a schematic diagram for describing a configuration example of a vehicle according to some embodiments;

FIG. 2 is a flowchart for describing an example of a visual field measurement method according to some embodiments;

FIGS. 3A and 3B are schematic diagrams for describing an example of a test case according to some embodiments;

FIGS. 4A to 4D are schematic diagrams for describing an example of a test case according to some embodiments;

FIGS. 5A and 5B are schematic diagrams for describing an example of a visual field according to some embodiments;

FIG. 6 is a diagram for describing an example of visual field information according to some embodiments;

FIG. 7 is a flowchart for describing an example of a driving assistance method according to some embodiments; and

FIG. 8 is a schematic diagram for describing an example of a driving assistance situation according to some embodiments.

### DESCRIPTION OF THE EMBODIMENTS

Hereinafter, embodiments will be described in detail with reference to the attached drawings. Note, the following embodiments are not intended to limit the scope of the claimed invention, and limitation is not made to an invention that requires a combination of all features described in the embodiments. Two or more of the multiple features described in the embodiments may be combined as appropriate. Furthermore, the same reference numerals are given to the same or similar configurations, and redundant description thereof is omitted.

### <Control Device and Application Example Thereof>

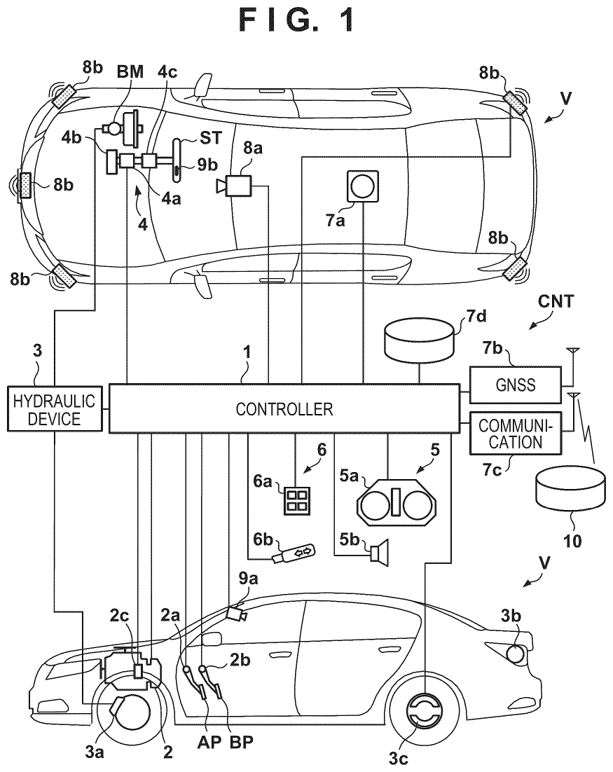

FIG. 1 is a block diagram of a control device CNT according to some embodiments, and is also a schematic diagram of a vehicle V, which is an application example thereof. In FIG. 1, an outline of the vehicle V is shown in a plan view and a side view. The vehicle V in the present embodiment is, as an example, a sedan-type four-wheeled passenger vehicle, and can be, for example, a parallel hybrid vehicle. The vehicle V is not limited to the four-wheeled passenger vehicle, and may be a straddle type vehicle (a two-wheeled or three-wheeled motorcycle) or a large-sized vehicle such as a truck or a bus.

The control device CNT includes a controller 1, which is an electronic circuit that performs control of the vehicle V, including driving assistance of the vehicle V. The controller 1 includes a plurality of electronic control units (ECUs). For example, an ECU is provided for each function of the control device CNT. Each ECU includes a processor represented by a central processing unit (CPU), a storage device such as a semiconductor memory, an interface with an external device, and the like. The storage device stores a program to be executed by the processor, data used for processing by the processor, and the like. The interface includes an input and output interface, and a communication interface. Each ECU may include a plurality of processors, a plurality of storage devices, and a plurality of interfaces. A program to be stored in the storage device may be installed in the control device CNT using a storage medium such as a CD-ROM so as to be stored in the storage device. Additionally or alternatively, the program to be stored in the storage device may be downloaded from an external server via wireless communication.

The controller 1 controls drive (acceleration) of the vehicle V by controlling a power unit (power plant) 2. The power unit 2 is a travelling drive unit that outputs a driving force for rotating driving wheels of the vehicle V, and can include an internal combustion engine, a motor, and an automatic transmission. The motor can be used as a drive source for accelerating the vehicle V, and can also be used as a generator at the time of deceleration or the like (regenerative braking).

In the present embodiment, the controller 1 controls outputs of the internal combustion engine and the motor, or switches a gear ratio of the automatic transmission in correspondence with a driver's drive operation detected by an operation detection sensor 2a provided in an accelerator pedal AP and an operation detection sensor 2b provided in a brake pedal BP, a vehicle speed of the vehicle V detected by a rotation speed sensor 2c, and the like. The automatic transmission is provided with the rotation speed sensor 2c that detects the rotation speed of an output shaft of the automatic transmission as a sensor that detects a traveling state of the vehicle V. It is possible to calculate the vehicle speed of the vehicle V from a detection result of the rotation speed sensor 2c.

The controller 1 controls braking (deceleration) of the vehicle V by controlling a hydraulic device 3. A driver's braking operation on the brake pedal BP is converted into hydraulic pressure in a brake master cylinder BM and transmitted to the hydraulic device 3. The hydraulic device 3 is an actuator capable of controlling a hydraulic pressure of a hydraulic oil supplied to a brake device 3a (for example, a disc brake device) provided on each of the four wheels based on the hydraulic pressure transmitted from the brake master cylinder BM.

The controller 1 can control braking of the vehicle V by performing drive control of an electromagnetic valve or the like included in the hydraulic device 3. The controller 1 can also configure an electric servo brake system by controlling the distribution of the braking force by the brake device 3a and the braking force by the regenerative braking of the motor included in the power unit 2. The controller 1 may turn on a brake lamp 3b at the time of braking.

The controller 1 controls steering of the vehicle V by controlling an electric power steering device 4. The electric power steering device 4 includes a mechanism for steering front wheels in response to a driver's drive operation (steering operation) on a steering wheel ST. The electric power steering device 4 includes a drive unit 4a that exerts a driving force (may be noted as steering assist torque) for assist in the steering operation or automatic steering of the front wheels of the vehicle V). The drive unit 4a includes a motor as a drive source. In addition, the electric power steering device 4 further includes a steering angle sensor 4b that detects a steering angle, and a torque sensor 4c that detects steering torque (also, referred to as steering load torque, and is distinguished from steering assist torque) borne by a driver.

The controller 1 controls electric parking brake devices 3c provided in respective rear wheels of the vehicle V. The electric parking brake device 3c includes a mechanism for locking the rear wheels. The controller 1 is capable of controlling locking and unlocking of the rear wheels by the electric parking brake device 3c.

The controller 1 controls an information output device 5 that notifies the inside of the vehicle of information. The information output device 5 includes, for example, a display device 5a that notifies the driver of information by images, and/or a sound output device 5b that notifies the driver of information by sound. Examples of the display device 5a include a display device provided in an instrument panel, and a display device provided in the steering wheel ST. In addition, the display device 5a may include a head-up display. The information output device 5 may notify an occupant of information by vibration or light.

The controller 1 receives an instruction input from the occupant (for example, the driver) via an input device 6. The input device 6 is disposed at a position operable by the driver, and includes, for example, a switch group 6a that is used when the driver gives an instruction for the vehicle V, and/or a blinker lever 6b for operating a direction indicator (blinker).

The controller 1 recognizes and determines a current position and a course (an attitude) of the vehicle V. In the present embodiment, the vehicle V includes a gyro sensor 7a, a global navigation satellite system (GNSS) sensor 7b, and a communication device 7c. The gyro sensor 7a detects a rotational motion (yaw rate) of the vehicle V. The GNSS sensor 7b detects a current position of the vehicle V. In addition, the communication device 7c performs wireless communication with a server that provides map information and traffic information, and acquires these pieces of information. Furthermore, the communication device 7c may read visual field information from a database 10. The visual field information is information to be used to estimate the visual field of the driver of the vehicle V. Details of the visual field information will be described later.

The controller 1 determines the course of the vehicle V, based on detection results of the gyro sensor 7a and the GNSS sensor 7b, also sequentially acquires map information about the course from the server via the communication device 7c, and stores the map information in a database 7d (a storage device). The vehicle V may also include another sensor for detecting a state of the vehicle V, such as an acceleration sensor for detecting acceleration of the vehicle V.

The controller 1 assists the driving of the vehicle V, based on detection results of various detection units provided in the vehicle V. The vehicle V includes a plurality of surrounding detection units 8a and 8b serving as an external sensor that detects the outside (surrounding situation) of the vehicle V, and a plurality of in-vehicle detection units 9a and 9b serving as an in-vehicle sensor that detects a state inside the vehicle (the state of occupants, particularly, the driver). The controller 1 can grasp the situation surrounding the vehicle V based on the detection results of the surrounding detection units 8a and 8b and then assist the driving of the vehicle V in correspondence with this surrounding situation. In addition, the controller 1 can determine whether the driver is performing a predetermined operation obligation imposed on the driver when assisting the driving based on the detection results of the in-vehicle detection units 9a to 9b.

The surrounding detection unit 8a is an imaging device (hereinafter, it may be referred to as a front camera 8a) that captures an image of the front of the vehicle V, and is attached to the vehicle interior side of the windshield at the front of the roof of the vehicle V, for example. The controller 1 can extract a contour of a target or a lane marking (such as a white line) on a road by analyzing an image captured by the front camera 8a.

The surrounding detection unit 8b is a millimeter wave radar (hereinafter, it may be referred to as a radar 8b), detects a target around the vehicle V using radio waves, and detects (measures) a distance to the target and a direction (azimuth) of the target with respect to the vehicle V. In the example shown in FIG. 1, five radars 8b are provided, one at the center of the front portion of the vehicle V, one at each of the left and right corner portions of the front portion, and one at each of the left and right corner portions of the rear portion.

The surrounding detection units provided in the vehicle V are not limited to the above configuration. The number of cameras and the number of radars may be changed. A light detection and ranging (LiDAR) for detecting a target around the vehicle V may be provided.

The in-vehicle detection unit 9a is an imaging device (hereinafter, it may be referred to as an in-vehicle camera 9a) that captures an image of the inside of the vehicle, and is attached to the vehicle interior side at the front of the roof of the vehicle V, for example. In the present embodiment, the in-vehicle camera 9a is a driver monitor camera that captures an image of the driver (for example, driver's eyes and face). The controller 1 can determine the direction of the line of sight and the face of the driver by analyzing an image (a face image of the driver) captured by the in-vehicle camera 9a.

The in-vehicle detection unit 9b is a grip sensor (hereinafter, referred it may be referred to as a grip sensor 9b) that detects the driver gripping the steering wheel ST, and is provided on, for example, at least a part of the steering wheel ST. As the in-vehicle detection units, a torque sensor 4c that detects the steering torque of the driver may be used.

### <Visual Field Measurement Method>

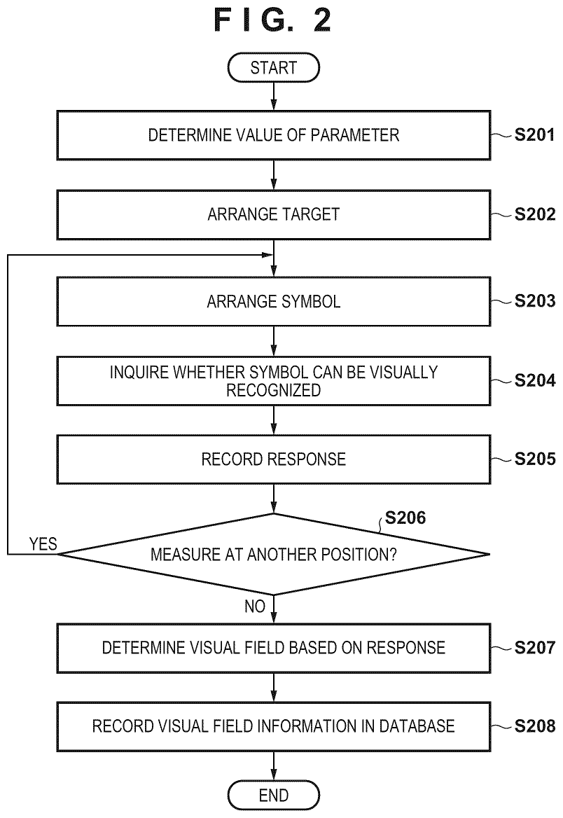

A method for measuring a visual field of a person according to some embodiments will be described with reference to FIG. 2. In the following description, a person whose visual field is to be measured is referred to as a subject, and a person who measures a visual field is referred to as a measurer. The method of FIG. 2 may be performed for each of a plurality of subjects. The visual field of one subject may be measured by a plurality of measurers.

In the method of FIG. 2, the visual field of a subject is measured in one test case. To start the method of FIG. 2, a measurer guides the subject to a fixed position of a measurement place. During the measurement of the visual field, the subject remains at the same position without moving from the fixed position. In this specification, when it is described that an object moves without specifying a criterion, it means that the object moves with respect to the ground. During the measurement of the visual field, the subject may stand upright or sit on a chair or the like. Alternatively, the visual field of the subject may be measured while the subject is on a moving body such as the vehicle V. As a result, the visual field of the subject is measured in a situation similar to driving a moving body. The moving body may be any moving body such as an automobile, a bicycle, or an electric wheelchair.

In S201, the measurer determines a value of a parameter to be used in a test case to be performed. The value of the parameter may be preset for each test case. The parameter may include at least one of (1) a distance from a subject to a target object, (2) a direction in which the target object is to be arranged with respect to the trunk of the subject, (3) a direction of the face of the subject with respect to the trunk of the subject, (4) a color of a symbol, (5) a light environment in which a visual field is to be measured, and (6) an index indicating the number of traffic participants to be arranged around the target object. Any of these parameters can affect the visual field of the subject. Each parameter will be described below.

(1) Distance from Subject to Target Object

The target object is a target that the subject continues to look at during the measurement of the visual field. The subject focuses on the target object during the measurement of the visual field. The target object may be any target that the subject can focus on. The visual field of the subject may vary depending on a focal length of the subject. Therefore, the test case may specify the distance from the subject to the target object. In the following description, the distance from the subject to the target object is referred to as a measurement target distance. The position of the subject may be the position of any one point of the subject, for example, the position between the eyebrows of the subject. The position of the target object may be the position of any one point of the target object, for example, the center of the target object.

(2) Direction in which Target Object is Arranged with Respect to Trunk of Subject

The visual field of the subject may vary depending on a direction of the target object with respect to the trunk of the subject. Therefore, the test case may specify the direction in which the target object is to be arranged with respect to the trunk of the subject. The direction of the target object with respect to the trunk of the subject may be defined by a combination of an elevation/depression angle and an azimuth angle in the direction of the target object with respect to the direction in front of the trunk of the subject. For example, the test case may specify arranging the target object in the direction in front of the trunk of the subject, arranging the target object in a direction at an angle of 45 degrees to the right of the trunk of the subject, or arranging the target object in a direction at an angle of 45 degrees to the upper of the trunk of the subject.

(3) Direction of Face of Subject with Respect to Trunk of Subject

The visual field of the subject may vary depending on a direction of the face of the subject with respect to the trunk of the subject. Therefore, the test case may specify the direction of the face of the subject with respect to the trunk of the subject. The direction of the face of the subject with respect to the trunk of the subject may be defined by a combination of an elevation/depression angle and an azimuth angle in the direction in front of the face of the subject with respect to the direction in front of the trunk. For example, the test case may specify that the subject directs the face to the front, that the subject directs the face at an angle of 45 degrees to the right, or that the subject directs the face at an angle of 45 degrees to the upper.

The direction of the target object with respect to the face of the subject is determined according to a relationship between the direction in which the target object is arranged with respect to the trunk of the subject and the direction of the face of the subject with respect to the trunk of the subject. The subject directs the line of sight to the direction of the target object with respect to the face of the subject. When the direction of the target object with respect to the trunk of the subject aligns the direction of the face of the subject with respect to the trunk of the subject, the subject looks at the target object with the pupils in the center. When the direction of the target object with respect to the trunk of the subject does not align the direction of the face of the subject with respect to the trunk of the subject, the subject looks at the target object with the pupils displaced from the center.

### (4) Color of Symbol

During the measurement of the visual field, a symbol is arranged around the target object to determine positions that the subject can visually recognize. The visual field of the subject may vary depending on the color of the symbol. Thus, the test case may specify the color of the symbol. The color of the symbol may be selected from a plurality of colors (for example, black, red, blue, yellow, and the like).

### (5) Light Environment

The visual field of the subject may vary depending on the light environment of the measurement place. Therefore, the test case may specify the light environment. The light environment may be an environment related to a light amount, a position of a light source, a color (wavelength) of light, and the like. For example, examples of light environment can include daytime, nighttime, backlighting, front lighting, specific weather (sunny, cloudy), and the like. The measurer may illuminate the measurement place with a light or the like to adjust the light environment.

### (6) Index Indicating the Number of Traffic Participants to be Arranged Around Target Object

The visual field of the subject may vary depending on the number of traffic participants included in the visual field of the subject. Therefore, the test case may specify an index indicating the number of traffic participants to be arranged around the target object. The traffic participant may include a pedestrian, a bicycle driver, a vehicle, or the like. The index indicating the number of traffic participants may be the number of traffic participants itself or a category of the number of traffic participants (for example, four categories of 0 people, 1 to 5 people, 6 to 10 people, and 11 or more people).

In S202, the measurer arranges the target object at the position designated by the value of the parameter determined in S201. Specifically, the measurer arranges the target object at the position in the designated direction with respect to the trunk of the subject and at the measurement target distance from the subject.

In S203, the measurer arranges the symbol at any position around the target object. The symbol has the color determined in S201. The symbol may be a two-dimensional symbol (for example, a circle, a square, a cross, a triangle, a specific character, or the like) or a three-dimensional symbol (for example, a sphere, a rectangular parallelepiped, a cone, or the like). The measurer may further arrange one or more traffic participants of the index determined in S201 around the target object.

In S204, the measurer instructs the subject to look at the target object (that is, to focus on the target object). The measurer may instruct the subject on the direction of the face based on the value of the parameter determined in S201. For example, the measurer may instruct the subject to look at the target object with the face directed in the front. Thereafter, the measurer inquires whether the subject can visually recognize the symbol while looking at the target object. When the central vision is measured as the visual field, the measurer may inquire whether the subject can visually recognize the presence of the symbol. When the effective visual field is measured as the visual field, the measurer may inquire whether the subject can visually recognize the type of symbol.

In S205, the measurer records the response from the subject (that is, whether or not the subject has been able to visually recognize the symbol) in association with the position of the symbol. The position of the symbol may be defined by a combination of an elevation/depression angle and an azimuth angle in the direction of the symbol with respect to the direction in front of the trunk of the subject and a distance from the trunk of the subject to the symbol. The position of the symbol may be the position of any one point of the symbol, for example, the center of the symbol.

In S206, the measurer determines whether the measurement is performed by arranging the symbol at another position. When it is determined that the measurement is performed by arranging the symbol at another position (“YES” in S206), the measurer repeats the steps of S203 to S205. When it is determined that the measurement is not performed by arranging the symbol at another position (“NO” in S206), the measurer performs S207. In this manner, the measurer arranges the symbol at a plurality of positions around the target object, and determines whether the subject can visually recognize the symbol at each position.

In S207, the measurer determines the visual field of the subject based on the record in S205 performed one or more times. Specifically, the measurer determines, as the visual field, a three-dimensional region including a position that the subject has responded that the subject was able to visually recognize and not including a position that the subject has responded that the subject was not able to visually recognize.

In S208, the measurer records visual field information in the database 10. The visual field information includes the value of the parameter used to measure the visual field and the visual field determined in S207. The parameter may include at least one of (1) the distance from the subject to the target object, (2) the color of the symbol, (3) the direction of the face of the subject with respect to the trunk, (4) the direction of the line of sight with respect to the face of the subject, (5) the light environment in which the measurement has been performed, and (6) the index indicating the number of traffic participants arranged around the target object.

The visual field of one subject may be measured in a plurality of test cases. In that case, the method of FIG. 2 may be performed for each of the test cases. The test cases may specify different values of parameters. For example, for each of a plurality of measurement target distances, the measurer may determine the visual field of the subject when the target object is arranged at a position at an individual measurement target distance from the subject. For each of a plurality of directions with respect to the trunk of the subject, the measurer may determine the visual field of the subject when the target object is arranged in an individual direction with respect to the trunk of the subject and the face of the subject is directed in the individual direction. For each of a plurality of directions with respect to the face of the subject, the measurer may determine the visual field of the subject when the target object is arranged in an individual direction with respect to the face of the subject and the line of sight of the subject is directed in the individual direction. The measurer may determine, for each of a plurality of light environments, the visual field of the subject in an individual light environment. For each of a plurality of colors, the measurer may determine the visual field of the subject when the symbol is represented in an individual color. The measurer may determine, for each of a plurality of indexes indicating the number of traffic participants, the visual field of the subject when traffic participants of an individual index are arranged.

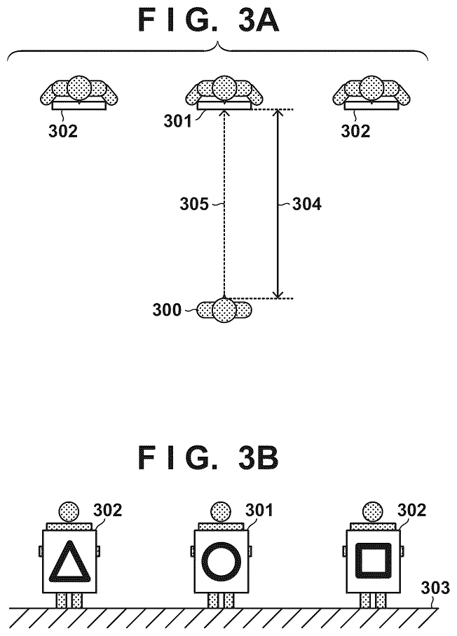

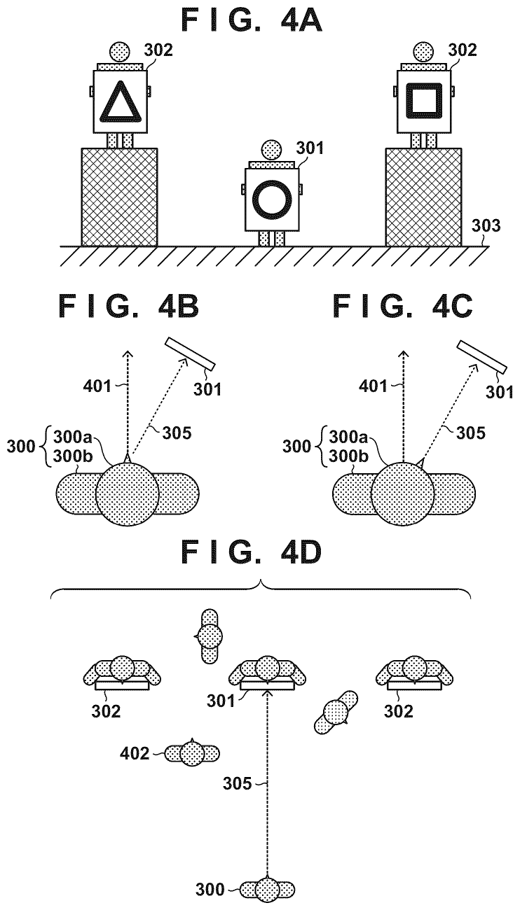

An example of a test case will be described with reference to FIGS. 3A to 4D. FIGS. 3A and 4B to 4D show a state in which the measurement place is viewed from above a subject 300. FIGS. 3B and 4A show the visual field of the subject 300. In the example of FIGS. 3A and 3B, a symbol (a circle in the example of FIG. 3B) drawn on a board 301 is used as the target object. The surface of the board 301 on which the symbol is drawn is, for example, a quadrangle having one side of 1 to 2 m. The board 301 is held by a measurer standing on a ground surface 303. Alternatively, the board 301 may be placed on a table. In addition, the symbol may be displayed on a display device instead of being drawn on the board 301. Furthermore, the symbol may be represented by the body of the measurer. For example, the symbol may be a particular pose of the measurer. The board 301 is arranged at a position at a measurement target distance 304 from the subject 300. During the measurement of the visual field, a line-of-sight direction 305 of the subject 300 is directed to the board 301.

In the example of FIGS. 3A and 3B, symbols (a triangle and square in the example of FIG. 3B) arranged around the target object in order to determine the position that the subject can visually recognize are drawn on boards 302. The surface on which each symbol is drawn is, for example, a quadrangle having one side of 1 to 2 m. The boards 302 are held by measurers standing on the ground surface 303. Alternatively, the boards 302 may be placed on tables. By representing the symbols with the boards 302, the visual field can be measured even in a place without a power supply or the like. The symbols may be displayed on display devices instead of being drawn on the boards 302. Furthermore, the symbol may be represented by the body of the measurer. For example, the symbol may be a particular pose of the measurer. The symbols each have a size that the subject can visually recognize when the symbols are in the visual field of the subject. As a result, the visual field can be measured without being affected by the eyesight of the subject.

The symbols used to determine the visual field are randomly selected so as not to be predicted by the subject. The symbols may be hidden from the subject until the subject directs the line of sight to the target object. For example, the measurer may invert the boards 302, or cover the symbols on the boards 302 with cloth or the like.

In the example of FIGS. 3A and 3B, in one execution of S202, two symbols (two boards 302) are arranged. Alternatively, in one execution of S202, only one symbol may be arranged, or three or more symbols may be arranged.

In the example of FIGS. 3A and 3B, the two symbols (boards 302) are arranged at symmetrical positions with the target object in the center. In addition, the two symbols (boards 302) are arranged on the same normal plane as the target object (board 301) with respect to the line-of-sight direction 305. Alternatively, the symbols may be arranged on a normal plane different from the target object (board 301) with respect to the line-of-sight direction 305.

In the example of FIGS. 3A and 3B, the two symbols (boards 302) are arranged at positions where the height from the ground surface 303 is the same as that of the target object (board 301). Alternatively, as shown in FIG. 4A, the height of the positions where the symbols are arranged may be different from the height of the position where the target object is arranged. The ground surface 303 is an example of a horizontal plane, and other horizontal planes may be used for height measurement. The symbols may be arranged directly above the target object.

In the test case shown in FIGS. 4B and 4C, the board 301 is arranged at a position deviated from a direction 401 in front of a trunk 300b of the subject 300. In the test case shown in FIG. 4B, the subject 300 is instructed to direct a face 300a in the direction 401 in front of the trunk 300b. Therefore, the subject 300 moves the pupils to direct the line-of-sight direction 305 to the symbol on the board 301. In the test case shown in FIG. 4C, the subject 300 is instructed to direct the face 300a to the symbol on the board 301. Therefore, the subject 300 directs the line-of-sight direction 305 together with the face toward the symbol on the board 301.

In the test case shown in FIG. 4D, three pedestrians 402 are arranged around the board 301 as traffic participants. The pedestrians 402 may walk around freely or remain at the same positions during the measurement of the visual field. Traffic participants other than pedestrians may be arranged, or traffic participants may not be arranged as shown in FIG. 3.

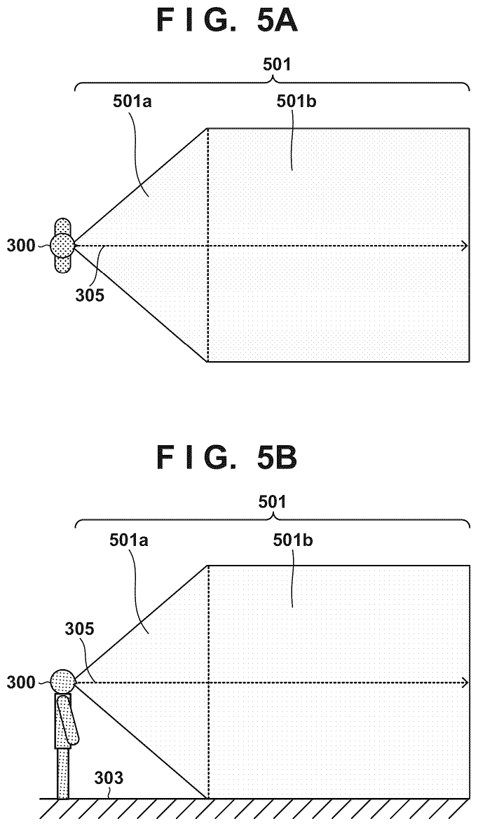

FIGS. 5A and 5B show an example of a visual field obtained by experiments by the inventors. FIG. 5A shows a visual field 501 of the subject 300 as viewed from above. FIG. 5B shows the visual field 501 of the subject 300 as viewed from the side. The visual field 501 has a shape formed by a conical portion 501a having the subject 300 as a vertex, and a cylindrical portion 501b extending from a bottom surface of the conical portion 501a.

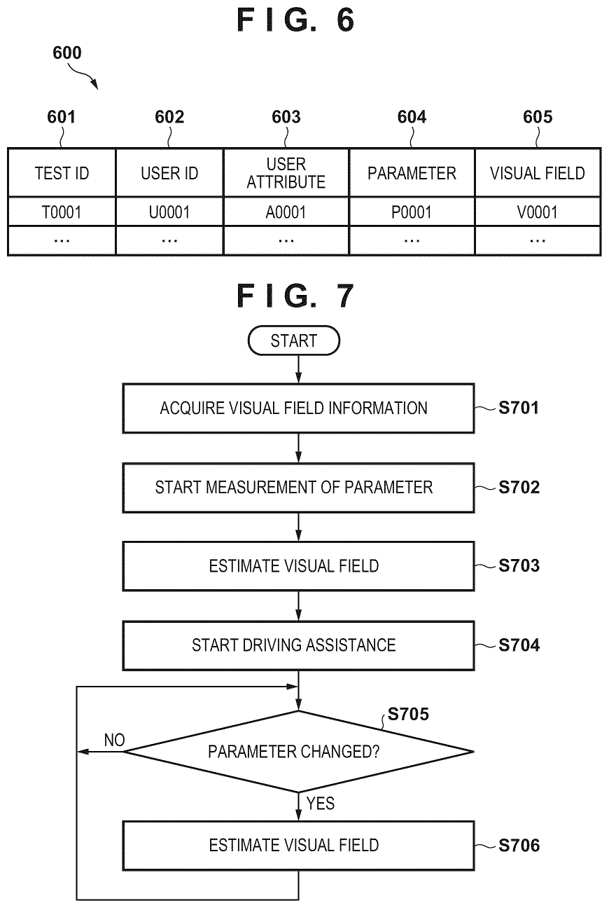

FIG. 6 shows an example of visual field information 600 recorded in the database 10 in S208 of FIG. 2. In FIG. 6, the visual field information 600 is recorded in a table format, but the visual field information 600 may be recorded in another format. The visual field information 600 has one entry for each execution of the method of FIG. 2. In the visual field information 600, information in columns 601 to 605 is recorded in association with each other. The column 601 stores identification information for identifying one test. The column 602 stores identification information for identifying a subject. The column 603 stores a value of an attribute of the subject. The measurer may record values of attributes that may affect the visual field as part of the visual field information 600. Such attributes may include, for example, at least one of an age category, an eyesight category, with or without glasses, with or without contact lenses, and with or without a specific disease (for example, glaucoma). The age category may be divided into 10-year increments or other granularity. The eyesight category may be divided into 0.1 increments or other granularity. The column 604 stores the value of the parameter used to measure the visual field. The column 605 stores the visual field determined in S207.

An administrator of the database 10 may integrate a plurality of entries in the visual field information 600 to generate a new entry. For example, the administrator may integrate a plurality of entries with the same attribute value (column 603) and the same parameter value (column 604) to generate a new entry. The columns 603 and 604 for the new entry store the information before the integration. The column 605 for the new entry stores a visual field representing the visual fields of the plurality of entries before the integration (for example, the union, intersection, or the like). The newly generated entry may be used as generic visual field information on a specific attribute value. Furthermore, the administrator may integrate a plurality of entries with the same the parameter values (column 604) to generate a new entry. The column 604 for the new entry stores the information before the integration. The column 605 for the new entry stores a visual field representing the visual fields of the plurality of entries before the integration (for example, the union, intersection, or the like). The newly generated entry may be used as general-purpose visual field information independent of the attributes.

### <Driving Assistance Method>

A driving assistance method to be performed by the vehicle V will be described with reference to FIG. 7. Each step of FIG. 7 may be performed by the control device CNT. Specifically, each step of FIG. 7 may be performed by a processor of the control device CNT executing a program that has been read into a memory of the control device CNT. Alternatively, some or all of the functional steps of FIG. 7 may be realized by a dedicated integrated circuit such as an application specific integrated circuit (ASIC). The method of FIG. 7 may be started in response to an instruction to start driving assistance from a driver of the vehicle V (hereinafter, simply referred to as a driver), or may be automatically started in response to the power of the vehicle V being turned on. The method of FIG. 7 may be performed by another moving body (for example, an airplane, a ship, or the like) other than the vehicle V.

In S701, the control device CNT acquires the visual field information 600 from the database 10. When the control device CNT can identify the driver and the visual field information 600 about this driver is recorded in the database 10, the control device CNT may acquire the visual field information 600 about this driver. In other cases, the control device CNT may acquire the general-purpose visual field information 600.

In S702, the control device CNT starts measurement of a parameter for estimating the visual field of the driver of the vehicle V. Thereafter, the control device CNT continues to measure the parameter. The parameter may include at least one of (1) a distance from a driver to a target positioned in a direction of the line of sight of the driver, (2) a color of a traffic participant positioned around the target, (3) a direction of the face of the driver with respect to the trunk, (4) a direction of the line of sight with respect to the face of the driver, (5) a light environment around the vehicle V, and (6) an index indicating the number of traffic participants positioned around the target.

The distance from the driver to the target positioned in the direction of the line of sight of the driver is measured based on, for example, detection results of the front camera 8a and the radar 8b. The color of the traffic participant positioned around the target positioned in the direction of the line of sight of the driver is measured based on, for example, a detection result of the front camera 8a. The direction of the face of the driver with respect to the trunk is measured based on, for example, a detection result of the in-vehicle camera 9a. The direction of the line of sight of the driver with respect to the face is measured based on, for example, a detection result of the in-vehicle camera 9a. The light environment around the vehicle V is measured based on, for example, detection results of the front camera 8a and the radar 8b. The index indicating the number of traffic participants positioned around the target positioned in the direction of the line of sight of the driver is measured based on, for example, detection results of the front camera 8a and the radar 8b.

In S703, the control device CNT estimates the visual field associated with the value of the parameter measured in S702 in the visual field information 600 as the visual field of the driver. If there is no visual field information 600 with a value matching the value of the parameter measured in S702, the control device CNT may determine the visual field information 600 with a value closest to the value of the parameter measured in S702. The visual field included in the visual field information 600 has the shape of the visual field 501 described in FIGS. 5A and 5B. Therefore, the control device CNT estimates the visual field of the driver so as to have a shape formed by a conical portion having the driver as a vertex and a cylindrical portion extending from the bottom surface of the conical portion.

In S704, the control device CNT provides driving assistance based on the visual field estimated in S703. Driving assistance may include notifying the driver of the presence of traffic participants. For example, when there is a possibility that a traffic participant positioned outside the visual field of the driver collides with the vehicle V, the control device CNT may notify the driver of the presence of the traffic participant. Alternatively or additionally, when the line of sight of the driver is not directed to a traffic participant although the traffic participant has been in the visual field of the driver for a predetermined time or more, the control device CNT may notify the driver of the presence of a traffic participant.

In S705, the control device CNT determines whether the value of the parameter for which measurement has been started in S702 has changed. When it is determined that the value of the parameter has changed (“YES” in S705), the control device CNT shifts the processing to S706, and in the other case (“NO” in S705), the processing repeats S705. In this manner, the control device CNT waits until the value of the parameter changes.

In S706, the control device CNT estimates the visual field associated with the changed value of the parameter as the visual field of the driver, similarly to S703. In this manner, the control device CNT updates the visual field to be used for driving assistance each time the value of the parameter changes.

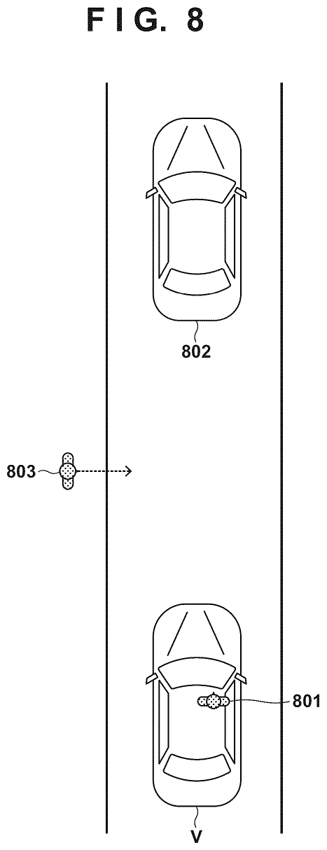

An example of a driving assistance situation will be described with reference to FIG. 8. It is assumed that a driver 801 of the vehicle V is traveling while watching a preceding vehicle 802. In this case, the target positioned in the direction of the line of sight of the driver is the preceding vehicle 802. Therefore, the control device CNT measures the distance from the driver 801 to the preceding vehicle 802. In addition, the control device CNT may determine that the face of the driver is directed to the front with respect to the trunk of the driver 801 and that the line of sight of the driver 801 is directed to the front with respect to the face. In addition, the control device CNT may determine that the light environment around the vehicle V is sunny in the daytime or that a traffic participant around the preceding vehicle 802 is only a pedestrian 803. Furthermore, the control device CNT may measure the color of the clothes of the pedestrian 803. The control device CNT may estimate the visual field of the driver 801 based on the values of these parameters and provide driving assistance based on this visual field. The color of the traffic participants can be different for each traffic participant. Therefore, the control device CNT may provide driving assistance for each traffic participant based on the visual field estimated for each traffic participant.

### SUMMARY OF EMBODIMENTS

[Item 1]A method for measuring a visual field (501) of a subject (300), the method comprising:
arranging (S202) a target object (301) at a position at a measurement target distance (304) from the subject and arranging a symbol (302) at a plurality of positions around the target object; and
determining (S207) the visual field of the subject based on a position of the plurality of positions at which the subject can visually recognize the symbol while looking at the target object.

According to this item, it is possible to measure the visual field according to a situation.

[Item 2] The method according to Item 1, wherein determining the visual field of the subject includes determining, for each of a plurality of measurement target distances, the visual field of the subject when the target object is arranged at a position at an individual measurement target distance from the subject.

According to this item, it is possible to measure the visual field in an individual situation.

[Item 3] The method according to Item 1 or 2, wherein the symbol has a size that the subject can visually recognize when the symbol is in the visual field of the subject.

According to this item, it is possible to accurately measure the visual field regardless of the eyesight of the subject.

[Item 4] The method according to any one of Items 1-3, wherein the plurality of positions includes a position having a height from a horizontal plane (303) different from a height of the target object.

According to this item, it is possible to measure the visual field in a three-dimensional space.

[Item 5] The method according to any one of Items 1-4, wherein determining the visual field of the subject includes, for each of a plurality of directions with respect to a trunk (300b) of the subject, determining the visual field of the subject when the target object is arranged in an individual direction with respect to the trunk of the subject and a face (300a) of the subject is directed in the individual direction.

According to this item, it is possible to measure the visual field in an individual situation.

[Item 6] The method according to any one of Items 1-5, wherein determining the visual field of the subject includes, for each of a plurality of directions with respect to a face of the subject, determining the visual field of the subject when the target object is arranged in an individual direction with respect to the face of the subject and a line of sight of the subject is directed in the individual direction.

According to this item, it is possible to measure the visual field in an individual situation.

[Item 7] The method according to any one of Items 1-6, wherein determining the visual field of the subject includes determining, for each of a plurality of light environments, the visual field of the subject in an individual light environment.

According to this item, it is possible to measure the visual field in an individual situation.

[Item 8] The method according to any one of Items 1-7, wherein determining the visual field of the subject includes determining, for each of a plurality of colors, the visual field of the subject when the symbol is represented in an individual color.

According to this item, it is possible to measure the visual field in an individual situation.

[Item 9] The method according to any one of Items 1-8, further comprising
arranging one or more traffic participants (402) around the target object, and
wherein determining the visual field of the subject includes determining, for each of a plurality of indices indicating of the number of the one or more traffic participants, the visual field of the subject when traffic participants of an individual index are arranged.

According to this item, it is possible to measure the visual field in an individual situation.

[Item 10] The method according to any one of Items 1-9, further comprising:
recording (S208) the determined visual field in a database (10) in association with a value of a parameter used in measurement, wherein
the parameter includes at least one of:
   a distance from the subject to the target object;
   a color of the symbol;
   a direction of a face of the subject with respect to a trunk during the measurement;
   a direction of a line of sight with respect to the face of the subject during the measurement;
   a light environment in which the measurement has been performed; and
   an index indicating the number of traffic participants arranged around the target object.

According to this item, it is possible to use the visual field measured in an individual situation.

[Item 11] The method according to any one of Items 1-10, wherein determining the visual field of the subject includes determining the visual field of the subject while the subject is on a moving body (V).

According to this item, it is possible to measure the visual field in a situation in which driving assistance is provided.

[Item 12]A moving body (V) configured to:
acquire visual field information from a database (10);
measure a parameter for estimating a visual field of a driver (801) of the moving body; and
estimate a visual field (501) associated with a value of the parameter in the visual field information as the visual field of the driver, wherein
the parameter includes at least one of.
   a distance from the driver to a target positioned in a direction of a line of sight of the driver;
   a color of a traffic participant positioned around the target;
   a direction of a face of the driver with respect to a trunk;
   a direction of the line of sight of the driver with respect to the face;
   a light environment around the moving body; and
   an index indicating the number of traffic participants positioned around the target.

According to this item, it is possible to accurately estimate the visual field to be used for driving assistance.

The invention is not limited to the foregoing embodiments, and various variations/changes are possible within the spirit of the invention.

What is claimed is:

1. A method for measuring a visual field of a subject, the method comprising:
arranging a target object at a position at a measurement target distance from the subject and arranging a symbol at a plurality of positions around the target object; and
determining the visual field of the subject based on a position of the plurality of positions at which the subject can visually recognize the symbol while looking at the target object.

2. The method according to claim 1, wherein determining the visual field of the subject includes determining, for each of a plurality of measurement target distances, the visual field of the subject when the target object is arranged at a position at an individual measurement target distance from the subject.

3. The method according to claim 1, wherein the symbol has a size that the subject can visually recognize when the symbol is in the visual field of the subject.

4. The method according to claim 1, wherein the plurality of positions includes a position having a height from a horizontal plane different from a height of the target object.

5. The method according to claim 1, wherein determining the visual field of the subject includes, for each of a plurality of directions with respect to a trunk of the subject, determining the visual field of the subject when the target object is arranged in an individual direction with respect to the trunk of the subject and a face of the subject is directed in the individual direction.

6. The method according to claim 1, wherein determining the visual field of the subject includes, for each of a plurality of directions with respect to a face of the subject, determining the visual field of the subject when the target object is arranged in an individual direction with respect to the face of the subject and a line of sight of the subject is directed in the individual direction.

7. The method according to claim 1, wherein determining the visual field of the subject includes determining, for each of a plurality of light environments, the visual field of the subject in an individual light environment.

8. The method according to claim 1, wherein determining the visual field of the subject includes determining, for each of a plurality of colors, the visual field of the subject when the symbol is represented in an individual color.

9. The method according to claim 1, further comprising
arranging one or more traffic participants around the target object, and
wherein determining the visual field of the subject includes determining, for each of a plurality of indices indicating of the number of the one or more traffic participants, the visual field of the subject when traffic participants of an individual index are arranged.

10. The method according to claim 1, further comprising:
recording the determined visual field in a database in association with a value of a parameter used in measurement, wherein
the parameter includes at least one of:
a distance from the subject to the target object;
a color of the symbol;
a direction of a face of the subject with respect to a trunk during the measurement;
a direction of a line of sight with respect to the face of the subject during the measurement;
a light environment in which the measurement has been performed; and
an index indicating the number of traffic participants arranged around the target object.

11. The method according to claim 1, wherein determining the visual field of the subject includes determining the visual field of the subject while the subject is on a moving body.

12. A moving body configured to:
acquire visual field information from a database;
measure a parameter for estimating a visual field of a driver of the moving body; and
estimate a visual field associated with a value of the parameter in the visual field information as the visual field of the driver, wherein
the parameter includes at least one of:
a distance from the driver to a target positioned in a direction of a line of sight of the driver;
a color of a traffic participant positioned around the target;
a direction of a face of the driver with respect to a trunk;
a direction of the line of sight of the driver with respect to the face;
a light environment around the moving body; and
an index indicating the number of traffic participants positioned around the target.
